(19) United States
(12) Patent Application Publication
(10) Pub. No.: US 2025/0009634 A1
(43) Pub. Date: Jan. 9, 2025

(54) LIQUID COMPOSITION OF CERAMIDES AND PALMITIC ACID AMIDES

(71) Applicant: Unifarco S.p.A., Santa Giustina BL (IT)

(72) Inventor: Giovanni Baratto, Santa Giustina (BL) (IT)

(73) Assignee: Unifarco S.p.A., Santa Giustina BL (IT)

(21) Appl. No.: 18/709,841

(22) PCT Filed: Nov. 18, 2022
(86) PCT No.: PCT/IB2022/061150

§ 371 Date: May 14, 2024

(30) Foreign Application Priority Data
Nov. 18, 2021 (IT) 102021000029171

(51) Int. Cl. A61K 8/68; A61K 8/06; A61K 8/42; A61K 8/44; A61Q 19/08

(52) U.S. Cl. CPC A61K 8/68; A61K 8/062; A61K 8/42; A61K 8/442; A61Q 19/08; A61K 2800/49; A61K 2800/522; A61K 2800/592

(57) ABSTRACT

Liquid composition consisting of: a) at least one ceramide b) at least one palmitic acid amide of formula (I) CH₃(CH₂)₁₄CONHR¹ (I) and c) di-(phytosteryl-2-octyldodecyl)-N-lauroyl-L-glutamate wherein R¹ is a straight or branched alkyl selected from the group consisting of a C1-C6 straight or branched alkyl optionally substituted with a hydroxyl group, a C4-C6 cycloalkyl, and wherein said components (a) and (b) are dissolved in said component (c).

### FIELD OF THE INVENTION

The present invention relates to an optimized liquid composition based on ceramides and palmitic acid amides and related stable and effective topical formulations in preventing and counteracting the skin inflammatory processes underlying skin diseases and/or inflamm-aging.

### BACKGROUND ART

Skin aging is an extremely heterogeneous physiological process.

In fact, numerous molecular mechanisms and pathways leading to cellular senescence can be identified.

In recent years, numerous studies on longevity have highlighted the role of chronic low-grade inflammation in aging, calling this phenomenon inflamm-aging [Baylis D, Bartlett D B, Patel H P, Roberts H C. Understanding how we age: insights into inflammaging. Longev Healthspan. 2013 May 2; 2 (1); Minciullo, P. L., Catalano, A., Mandraffino, G. et al. Inflammaging and Anti-Inflammaging: The Role of Cytokines in Extreme Longevity. Arch. Immunol. Ther. Exp. 64, 111-126 (2016).]

According to recent scientific evidence, inflamm-aging is mainly involved in the development of neurological and cardiovascular diseases but can affect the cells of all organs, including the skin.

Inflamm-aging is established and spread at the cellular level and is a silent chronic inflammatory state that occurs when cells lose their ability in regulating inflammatory and defence processes from internal and external aggressions.

The causes for inflamm-aging are:
internal: increase in free radicals due to physical and mental stress, unbalanced diet, chronic intake of drugs and hormonal therapies;
external: UV rays, environmental pollution, lifestyle.

The consequences are an accelerated onset of the typical signs of skin aging:
dryness;
spots or discolorations;
wrinkles;
loss of tone and elasticity;
redness;
scaling;
opacity;
burning sensation or skin tightness

In addition to the ongoing inflammatory process, the skin, in particular sensitive skin, has a poor defence capacity, which is why it is physiologically more fragile and subject to alterations of the epidermal barrier. This is why it is more likely to develop and self-fuel inflamm-aging processes. The reinforcement of the barrier with a consequent reduction of inflammation and therefore of the release of pro-inflammatory cytokines can be another mechanism to be evaluated to combat skin aging.

The use of ceramide-based products helps in counteracting skin aging as it acts effectively on the skin by restructuring and protecting the epidermal barrier.

At the state of the art there are numerous cosmetic ingredients that are effective in actively counteracting the physiological process of skin aging. The term endocannabinoids refers to a heterogeneous family of amides and esters of polyunsaturated fatty acids found in various human tissues. Arachidonic acid ethanolamide (Anandamide, ANA) and 2-arachidonylglycerol are certainly the two most described derivatives in the literature [Sugiura T, Kobayashi Y, Oka S, Waku K. Biosynthesis and degradation of anandamide and 2-arachidonoylglycerol and their possible physiological significance. Prostaglandins Leukot Essent Fatty Acids 66:173-192]

These endogenous derivatives bind with different affinities to the known cannabinoid receptors CB1 and CB2 and the effect of ANA has been shown to be dependent on its extracellular concentration, which is controlled in addition to the enzymatic system responsible for its synthesis, also by an intracellular transport system and an enzymatic degradation system (Fatty Acid Hydrolases, FAAH).

N-isopropyl-palmitoyl amide and palmitoylethanolamide are endocannabinoid-like molecules that can play a key role in balancing important inflammatory mediators and inflammatory-aging.

Phytosteryl/Octyldodecyl Lauroyl Glutamate (INCI) of formula IUPAC (di-(phytosteryl-2-octyldodecyl)-N-lauroyl-L-glutamate) is an active ingredient for the formulation of products for topical use, and is an eudermic oil with a chemical structure similar to the components of the epidermal barrier, in particular to intercellular lipids.

Formulations of topical products (e.g. cosmetics or medical devices) containing ceramides and/or endocannabinoid-like derivatives may be very effective in counteracting the inflammatory processes underlying altered barrier physiology. However, they are all poorly soluble ingredients and are known to have stability and crystallization concerns.

The formulation of cosmetics with poorly soluble and very unstable active ingredients therefore poses technical challenges to formulators, such as the instability of the ingredient, dispersion problems, quality and safety.

The main problem consists in the solubilisation of this raw material in the final formulation, requiring careful and continuous control in the production phases of industrial scale-up.

To overcome the problem, it is possible to act on the production method, varying the order of the stages of addition of raw materials; however, the heating process at high temperatures, normally necessary to dissolve waxes, for example, could cause the degradation of other raw materials that make up the formula. A good compromise may be the pre-solution with a solvent.

However, the selection of the right solvent that leads to the formulation of a product for safe, stable and effective topical use requires careful evaluation, on the part of the formulator, of several factors:
solvent behaviour in the base (vehicle)
tolerability after application (also linked to the peculiar characteristics of the application site). (For example, tissue inflamed with dermatitis).
compliance by the end user. (The formulated product must be pleasant and reused several times).

JP5432470B2 discloses an oily composition containing ceramide dissolved in a mixture of a triglyceride of a fatty acid and a phytosteryl derivative.

EP 3125865 discloses a composition comprising ceramide and palmitic acid amide with monoethylamine dispersed as such in an oil-in-water emulsion together with other active ingredients and/or excipients.

In the online MINTEL GNPD database dated 20 Jan. 2020 XP055926288 database accession no. 7199719 and dated 1 Jan. 2017 XP002781544, two Vecua creams and night cream comprising a plethora of components including various types of ceramide ceramide NP and cetyl PG hydroxyethylpalmitamide are disclosed.

Semenzato et al in “A New Endocannabinoid as Anti-Inflammatory Cosmetic Active: An In vitro study on Reconstructed Skin Model” CURR. UPDATES DERMATOL. PROBL. Vol. 2019, number 1, 3 Jan. 2019.XP055036765 disclose that a new cannabinoid, the isopalmide, on a reconstructed skin model exhibits anti-inflammatory properties.

On the Unifarco Biomedical website, a Ceramage® branded day cream containing among the several elements ceramide, isopalmide and (di-(phytosteryl-2-octyldodecyl)-N-lauroyl-L-glutamate) is disclosed.

Ceramide and isopalmide (di-(phytosteryl-2-octyldodecyl)-N-lauroyl-L-glutamate) are dispersed in the commercial formulation directly and not in the form of a liquid composition.

### SUMMARY OF THE INVENTION

The applicant has now surprisingly found that both ceramides and palmitic acid amides can be dissolved in a single step by mixing with the Phytosteryl/Octyldodecyl Lauroyl Glutamate (INCI) of formula IUPAC (di-(phytosteryl-2-octyldodecyl)-N-lauroyl-L-glutamate), which is able to fully solubilise the two components and thus contributes to creating a stable liquid composition suitable for inclusion in compositions for topical use.

The object of the present invention is therefore a liquid composition consisting of:
a) at least one ceramide
b) at least one palmitic acid amide of formula (I)

CH₃(CH₂)₁₄CONHR¹ (I)

and
c) di-(phytosteryl-2-octyldodecyl)-N-lauroyl-L-glutamate wherein R¹ is a straight or branched alkyl selected from the group consisting of a C1-C6 straight or branched alkyl optionally substituted with a hydroxyl group, a C4-C6 cycloalkyl, and wherein said components (a) and (b) are dissolved in said component (c)

### DESCRIPTION OF THE FIGURES

FIG. 1 depicts the chemical formulas of some of the ceramides that may be used in the composition according to the present invention.

FIG. 2 shows the photos of the solubilisation tests of 0.3% Ceramide 3 in different oils.

FIG. 3 shows the photos of the solubilisation tests of 0.3% N-isopropyl-palmitoyl amide (Isopalmide) in different oils.

FIG. 4 shows the photos of the solubilisation tests of 0.3% Palmitoylethanolamide (Palmitamide MEA or PMEA) in different oils.

FIG. 5 shows the photos of the solubilisation tests of the liquid composition subject-matter of the patent, at increasing concentrations of N-isopropyl-palmitoyl amide (Isopalmide) and Ceramide 3.

FIG. 6 shows the photos of the solubilisation tests of the liquid composition subject-matter of the patent, at increasing concentrations of Palmitoylethanolamide (PMEA) and Ceramide 3.

FIG. 7 shows the photos of the solubilisation tests of the liquid composition subject-matter of the patent, at increasing concentrations of N-isopropyl-palmitoyl amide (Isopalmide), Palmitoylethanolamide (PMEA) and Ceramide 3.

FIG. 8 show photos of the mixture of isopalmide, ceramide and di-(phytosteryl-2-octyldodecyl)-N-lauroyl-L-glutamate in the weight ratios between them in the Ceramage cosmetic formulations.

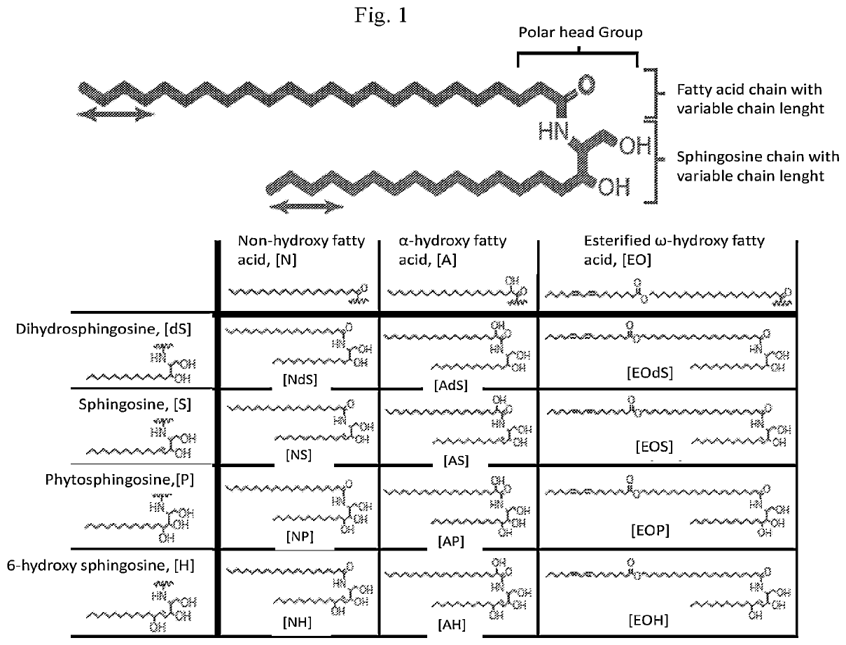

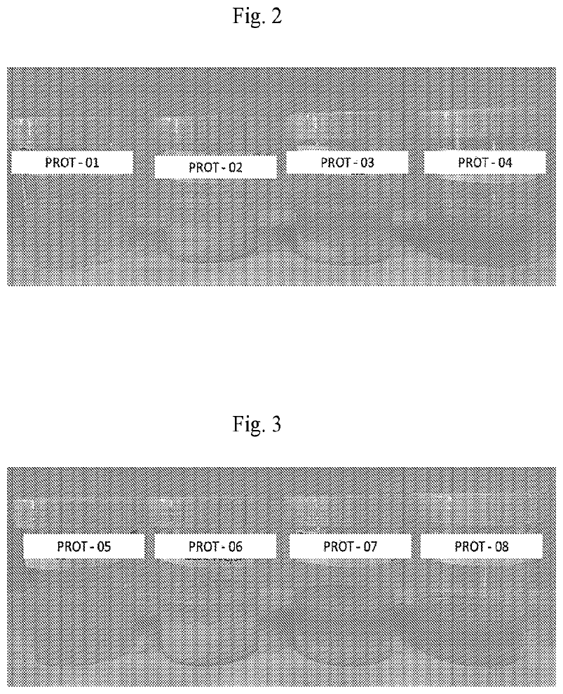

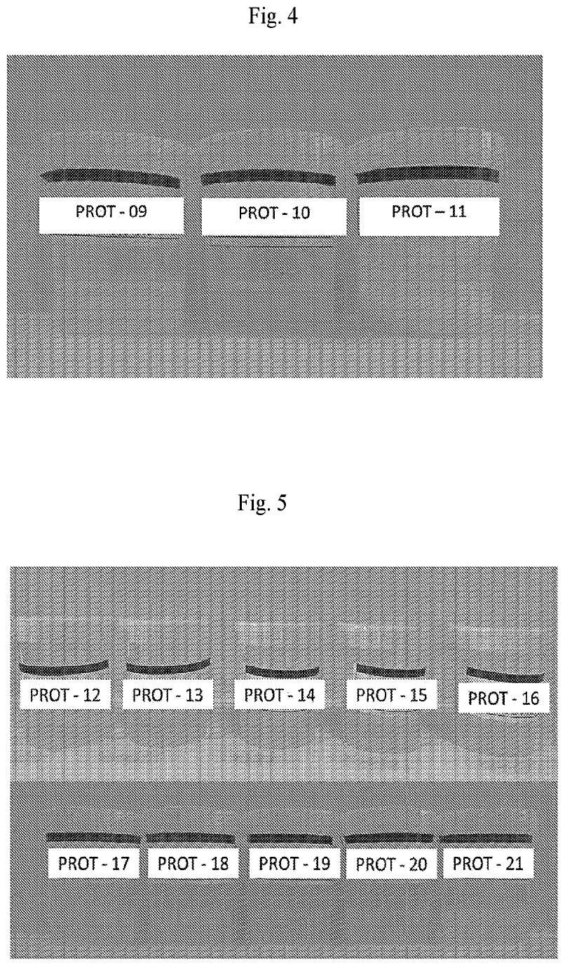

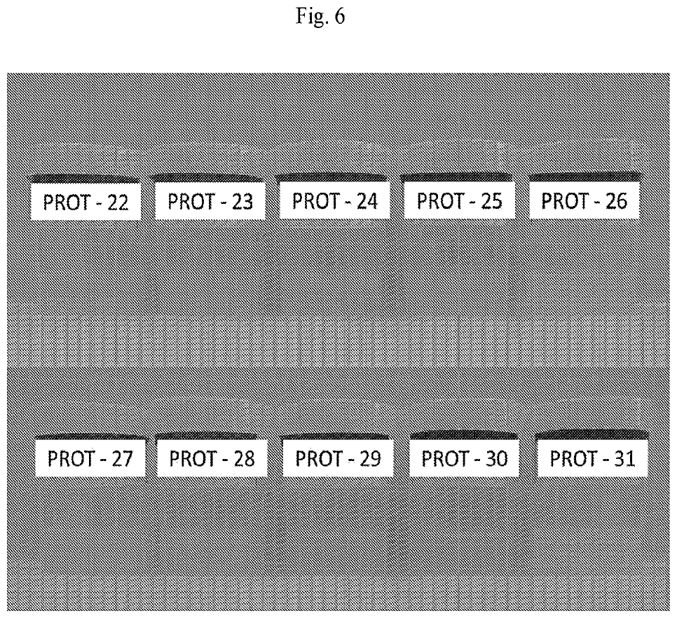

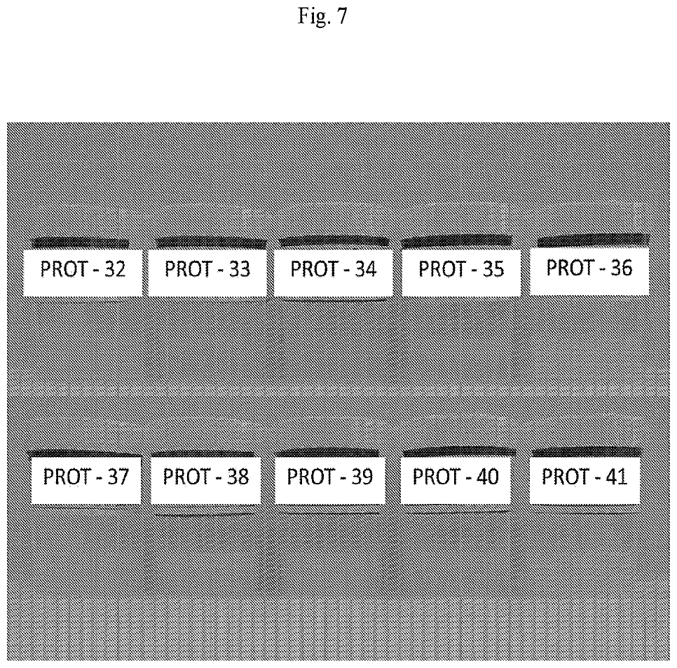

### DETAILED DESCRIPTION OF THE INVENTION

For the purposes of the present invention, the definitions “comprising” and “containing” provide for the possibility of further components in addition to those mentioned after said definition.

Conversely, for the purposes of the present invention the definition “consisting of” excludes the possibility of further components other than those listed after said definition.

In the present invention there is described a liquid composition consisting of:
(a) at least one ceramide,
(b) at least one palmitic acid amide of formula (I)

CH₃(CH₂)₁₄CONHR¹ (I)
(c) di-(phytosteryl-2-octyldodecyl)-N-lauroyl-L-glutamate wherein said components (a) and (b) are dissolved

For the purposes of the present invention it is specified that, in the palmitic acid amide of formula (I), R¹ is a linear or branched alkyl selected from the group consisting of a C1-C6 linear or branched alkyl optionally substituted with a hydroxyl group, a C4-C6 cycloalkyl. In particular, R¹ is selected from: n-propyl, isopropyl, n-butyl, -n-pentyl, iso-pentyl, ethylene-hydroxy, cyclobutyl, cyclopentyl and cyclohexyl.

Preferably the palmitic acid amides of formula (I) are selected from N-isopropyl-palmitoyl amide and palmitoylethanolamide or a mixture thereof.

In the liquid composition subject-matter of the present invention the ceramide or component a) is selected from N-acetylfingosine (ceramide 2 or NS) or the amide of phytosphingosine with stearic acid (Ceramide 3 or NP), preferably it is the amide of phytosphingosine with stearic acid.

For the purposes of the present invention the weight ratio of the components a) and b) is comprised between 1:2 and 2:1, preferably said weight ratio of the components (a) and (b) is 1:1.

Preferably when in the liquid composition subject-matter of the present invention the component b) contains both the isopalmide and the PMEA in a weight ratio between 1:2 and 2:1 more preferably 1:1.

For the purposes of the present invention the components a) and b) are present at concentrations less than or equal to 7%, preferably comprised between 0.05 and 6%, more preferably between 0.05 and 2%, still more preferably between 0.1 and 1% by weight on the total weight of said association.

The liquid composition described in the present invention can be inserted into topical use formulations wherein said liquid composition is contained in amounts comprised between 0.5 and 10% more preferably between 1 and 5% by weight on the total weight of said topical formulation.

The topical formulation comprising the liquid composition subject-matter of the present invention may be in the form of an oil-in-water emulsion and in this case is substantially hydrophilic, or may also be a water-in-oil emulsion and thus be substantially lipophilic, or an anhydrous formulation thus lipophilic.

Topical formulations comprising the liquid composition described in the present invention can be used in treating and preventing skin inflammatory processes, in particular those related to inflammation-aging.

By skin inflammatory process, the Applicant also refers to the one developed by and/or associated with skin diseases such as: Atopic dermatitis (DIC, DAC), psoriasis, seborrhoeic dermatitis, acne, rosacea, actinic keratosis and ichthyosis.

Below is the procedure followed by the Applicant which allowed him to achieve at the present invention.

### 1—Selection of the Solubilising Agent: Prototype Preparation

The samples were made in the standard quantity of 50 g, sufficient to perform the solubilisation and stability tests. The concentration ranges of the investigated actives and the sample preparation methods were established after a critical reading of the technical data sheets of each raw material. The initial purpose of this work was to identify a solubilising agent for some raw materials such as Ceramide, N-isopropyl-palmitoyl amide (isopalmide) and palmitoylethanolamide (Palmitamide MEA or PMEA) that in the daily practice of a formulator exhibit problems of solubilisation.

To evaluate the stability over time of the prepared samples, the centrifuge test was performed, which is a method that allows to detect any instability effects such as sedimentation and phase separation following a condition of strong mechanical stress.

Table I shows the names of each prototype with the relative concentration of Ceramide, Isopalmide and PMEA and the different raw materials used initially as screening.

TABLE 1
Systematic for the choice of the solubilising agent

| | ACTIVE | | Oper. | Appearance | appearance | Post |
|---|---|---|---|---|---|---|
| PROTOTYPE | % by | Solubilising Oil | solubilis. | after | after 1 | centrifuge |
| NAME | weight | (INCI name) | cond. | solubilisation | day | appearance |
| PROT - 01 | ceramide -3 | Squalane | Heat. up to | solution, | suspension | separated |
| | 0.3% | | 110° C. | translucent | and opaque | |
| | | | | solution | solution | |
| PROT - 02 | ceramide -3 | Phytostery lmacadamate/ | Heat. up to | opaque but | separated | separated |
| | 0.3% | squalane | 110° C. | homogeneous | opaque | |
| | | 50/50 (w/w) | | solution | solution | |
| | | | | | with deposit | |
| PROT - 03 | ceramide -3 | Phytosteryl/Octyldodecyl | Heat. up to | clear | separated | separated |
| | 0.3% | Lauroyl Glutamate/ | 110° C. | transparent | solution | |
| | | Phytosteryl/macadamate = | | yellow | | |
| | | 50:50 w/w | | solution | | |
| PROT - 04 | ceramide -3 | Phytosteryl/Octyldodecyl | Heat. up to | clear yellow | opaque | opaque |
| | 0.3% | Lauroyl Glutamate | 110° C. | solution | yellow | yellow |
| | | | | | solution | solution |
| PROT - 05 | Isopalmide | Squalane | Heat. up to | transparent | opaque | separated |
| | 0.3% | | 70° C. | solution | solution | |
| PROT - 06 | Isopalmide | Phytosteryl macadamate/ | Heat. up to | transparent | separated | separated |
| | 0.3% | squalane = | 70° C. | solution | solution | |
| | | 50/50 w/w | | | with deposit | |
| PROT - 07 | Isopalmide | Phytosteryl/Octyldodecyl | Heat. up to | yellow | separated | separated |
| | 0.3% | Lauroyl | 70° C. | transparent | solution | |
| | | Glutamate:Phytosteryl | | clear solution | with deposit | |
| | | macadamiate = | | | | |
| | | 50:50 (w/w) | | | | |
| PROT - 08 | Isopalmide | Phytosteryl/Octyldodecyl | Heat. up to | clear yellow | clear light | clear light |
| | 0.3% | Lauroyl Glutamate | 70° C. | solution | yellow | yellow |
| | | | | | solution | solution |
| PROT - 09 | PMEA | Squalane | Heat. up to | transparent | opaque | deposit on |
| | 0.3% | | 110° C. | solution | yellow | the bottom |
| | | | | | solution | |
| PROT - 10 | PMEA | Phytosteryl/Octyldodecyl | Heat. up to | transparent | very clear | very clear |
| | 0.3% | Lauroyl Glutamate | 110° C. | sol. | yellow | yellow |
| | | | | | solution | solution |
| PROT - 11 | PMEA | Phytosteryl macadamiate | Heat. up to | transparent | opaque | separated |
| | 0.3% | | 110° C. | solution | yellow | |
| | | | | | solution | |

As can be observed by just using Phytosteryl/Octyldodecyl Lauroyl Glutamate as solubilising agent it is possible to obtain the individual solubilisation of Ceramide 3, isopalmide and PMEA.

### 2—Systematics of Analyses of the Mixtures

After having unexpectedly identified that it was possible to use a single solubilising agent common to all the raw materials under study (ceramides and palmitic acid amides), systematics were set up to analyse the behaviour of the solution containing combinations of ceramides and palmitic acid amides, varying both the combination and the concentration of each component of the liquid composition subject-matter of the patent.

TABLE 2
Systematics of Ceramide 3 with Isopalmide at increasing concentrations,
solubilised in Phytosteryl/Octyldodecyl Louroyl Glutamate

| | | | | Oper. | appearance | appearance | Post |
|---|---|---|---|---|---|---|---|
| PROTOTYPE | Solubilising Oil | | | Solubilisation | after | after 1 | centrifuge |
| NAME | (INCI name) | ceramide -3 | isopalmide | cond. | solubilisation | day | appearance |
| PROT - | Phytosteryl/ | 0.10% | 0.10% | Heat. up | transparent | opaque | opaque |
| 12 | Octyldodecyl | | | to 110° C. | solution | yellow | yellow |
| | Lauroyl | | | | | solution | solution |
| | Glutamate | | | | | | |
| PROT - | Phytosteryl/ | 0.20% | 0.20% | heating up | clear yellow | opaque | opaque |
| 13 | Octyldodecyl | | | to 110° C. | solution | yellow | yellow |
| | Lauroyl | | | | | solution | solution |
| | Glutamate | | | | | | |
| PROT - | Phytosteryl/ | 0.30% | 0.30% | heating up | clear yellow | opaque | opaque |
| 14 | Octyldodecyl | | | to 110° C. | solution | yellow | yellow |
| | Lauroyl | | | | | solution | solution |
| | Glutamate | | | | | | |
| PROT - | Phytosteryl/ | 0.40% | 0.40% | Heat. up | clear yellow | opaque | opaque |
| 15 | Octyldodecyl | | | to 110° C. | solution | yellow | yellow |
| | Lauroyl | | | | | solution | solution |
| | Glutamate | | | | | | |
| PROT - | Phytosteryl/ | 0.50% | 0.50% | Heat. up | clear yellow | opaque | opaque |
| 16 | Octyldodecyl | | | to 110° C. | solution | yellow | yellow |
| | Lauroyl | | | | | solution | solution |
| | Glutamate | | | | | | |
| PROT - | Phytosteryl/ | 0.60% | 0.60% | Heat. up | clear yellow | opaque | opaque |
| 17 | Octyldodecyl | | | to 110° C. | solution | yellow | yellow |
| | Lauroyl | | | | | solution | solution |
| | Glutamate | | | | | | |
| PROT - | Phytosteryl/ | 0.70% | 0.70% | Heat. up | clear yellow | opaque | opaque |
| 18 | Octyldodecyl | | | to 110° C. | solution | yellow | yellow |
| | Lauroyl | | | | | solution | solution |
| | Glutamate | | | | | | |
| PROT - | Phytosteryl/ | 0.80% | 0.80% | Heat. up | clear yellow | opaque | opaque |
| 19 | Octyldodecyl | | | to 110° C. | solution | yellow | yellow |
| | Lauroyl | | | | | solution | solution |
| | Glutamate | | | | | | |
| PROT - | Phytosteryl/ | 0.90% | 0.90% | Heat. up | clear yellow | opaque | opaque |
| 20 | Octyldodecyl | | | to 110° C. | solution | yellow | yellow |
| | Lauroyl | | | | | solution | solution |
| | Glutamate | | | | | | |
| PROT - | Phytosteryl/ | 1.00% | 1.00% | Heat. up | clear yellow | opaque | opaque |
| 21 | Octyldodecyl | | | to 110° C. | solution | yellow | yellow |
| | Lauroyl | | | | | solution | solution |
| | Glutamate | | | | | | |

TABLE 3
Systematics of Ceramide 3 with PMEA at increasing concentrations,
solubilised in Phytosteryl/Octyldodecyl Lauroyl Glutamate

| | | | | | appearance | appearance | Post |
|---|---|---|---|---|---|---|---|
| PROTOTYPE | Solubilising Oil | | | | after | after 1 | centrifuge |
| NAME | (INCI name) | ceramide -3 | pmea | observations | solubilisation | day | appearance |
| PROT - 22 | Phytosteryl/ | 0.10% | 0.10% | Heat. up to | transparent | semi- | semi- |
| | Octyldodecyl | | | 110° C. | solution | transp. | transp. |
| | Lauroyl Glutamate | | | | | solution | solution |
| | | | | | | yellow | yellow |
| PROT - 23 | Phytosteryl/ | 0.20% | 0.20% | Heat. up to | transparent | yellow | yellow |
| | Octyldodecyl | | | 110° C. | solution | opaque | opaque |
| | Lauroyl Glutamate | | | | | solution | solution |
| PROT - 24 | Phytosteryl/ | 0.30% | 0.30% | Heat. up to | transparent | yellow | yellow |
| | Octyldodecyl | | | 110° C. | solution | opaque | opaque |
| | Lauroyl Glutamate | | | | | solution | solution |
| PROT - 25 | Phytosteryl/ | 0.40% | 0.40% | Heat. up to | transparent | yellow | yellow |
| | Octyldodecyl | | | 110° C. | solution | opaque | opaque |
| | Lauroyl Glutamate | | | | | solution | solution |
| PROT - 26 | Phytosteryl/ | 0.50% | 0.50% | Heat. up to | transparent | yellow | yellow |
| | Octyldodecyl | | | 110° C. | solution | opaque | opaque |
| | Lauroyl Glutamate | | | | | solution | solution |
| PROT - 27 | Phytosteryl/ | 0.60% | 0.60% | Heat. up to | transparent | yellow | yellow |
| | Octyldodecyl | | | 110° C. | solution | opaque | opaque |
| | Lauroyl Glutamate | | | | | solution | solution |
| PROT - 28 | Phytosteryl/ | 0.70% | 0.70% | Heat. up to | transparent | yellow | yellow |
| | Octyldodecyl | | | 110° C. | solution | opaque | opaque |
| | Lauroyl Glutamate | | | | | solution | solution |
| PROT - 29 | Phytosteryl/ | 0.80% | 0.80% | Heat. up to | transparent | yellow | yellow |
| | Octyldodecyl | | | 110° C. | solution | opaque | opaque |
| | Lauroyl Glutamate | | | | | solution | solution |
| PROT - 30 | Phytosteryl/ | 0.90% | 0.90% | Heat. up to | transparent | yellow | yellow |
| | Octyldodecyl | | | 110° C. | solution | opaque | opaque |
| | Lauroyl Glutamate | | | | | solution | solution |
| PROT - 31 | Phytosteryl/ | 1.00% | 1.00% | Heat. up to | transparent | yellow | yellow |
| | Octyldodecyl | | | 110° C. | solution | opaque | opaque |
| | Lauroyl Glutamate | | | | | solution | solution |

TABLE 4
Systematics with Ceramide 3, Isopalmide and PMEA at increasing concentrations,
solubilised in Phytosteryl/Octyldodecyl Lauroyl Glutamate

| | | | | | Oper. | appearance | appearance | Post |
|---|---|---|---|---|---|---|---|---|
| PROTOTYPE | Solubilising Oil | | | | Solubilisation | after | after 1 | centrifuge |
| NAME | (INCI name) | ceramide -3 | isopalmide | pmea | cond. | solubilisation | day | appearance |
| PROT - 32 | Phytosteryl/ | 0.10% | 0.10% | 0.10% | Heat. up to | transparent | semi- | semi- |
| | Octyldodecyl | | | | 110° C. | solution | transparent | transparent |
| | Lauroyl Glutamate | | | | | | yellow | yellow |
| | | | | | | | solution | solution |
| PROT - 33 | Phytosteryl/ | 0.20% | 0.20% | 0.20% | Heat. up to | transparent | opaque | opaque |
| | Octyldodecyl | | | | 110° C. | solution | yellow | yellow |
| | Lauroyl Glutamate | | | | | | solution | solution |
| PROT - 34 | Phytosteryl/ | 0.30% | 0.30% | 0.30% | Heat. up to | transparent | opaque | opaque |
| | Octyldodecyl | | | | 110° C. | solution | yellow | yellow |
| | Lauroyl Glutamate | | | | | | solution | solution |
| PROT - 35 | Phytosteryl/ | 0.40% | 0.40% | 0.40% | heating up | transparent | opaque | opaque |
| | Octyldodecyl | | | | to 110° C. | solution | yellow | yellow |
| | Lauroyl Glutamate | | | | | | solution | solution |
| PROT - 36 | Phytosteryl/ | 0.50% | 0.50% | 0.50% | Heat. up to | transparent | opaque | opaque |
| | Octyldodecyl | | | | 110° C. | solution | yellow | yellow |
| | Lauroyl Glutamate | | | | | | solution | solution |
| PROT - 37 | Phytosteryl/ | 0.60% | 0.60% | 0.60% | Heat. up to | transparent | opaque | opaque |
| | Octyldodecyl | | | | 110° C. | solution | yellow | yellow |
| | Lauroyl Glutamate | | | | | | solution | solution |
| PROT - 38 | Phytosteryl/ | 0.70% | 0.70% | 0.70% | Heat. up to | transparent | opaque | opaque |
| | Octyldodecyl | | | | 110° C. | solution | yellow | yellow |
| | Lauroyl Glutamate | | | | | | solution | solution |
| PROT - 39 | Phytosteryl/ | 0.80% | 0.80% | 0.80% | Heat. up to | transparent | opaque | opaque |
| | Octyldodecyl | | | | 110° C. | solution | yellow | yellow |
| | Lauroyl Glutamate | | | | | | solution | solution |
| PROT - 40 | Phytosteryl/ | 0.90% | 0.90% | 0.90% | Heat. up to | transparent | opaque | opaque |
| | Octyldodecyl | | | | 110° C. | solution | yellow | yellow |
| | Lauroyl Glutamate | | | | | | solution | solution |
| PROT - 41 | Phytosteryl/ | 1.00% | 1.00% | 1.00% | Heat. up to | transparent | opaque | opaque |
| | Octyldodecyl | | | | 110° C. | solution | yellow | yellow |
| | Lauroyl Glutamate | | | | | | solution | solution |

For illustrative purposes, the following cosmetic formulations are reported in which the liquid compositions according to the present invention.

Formulation 1 O/W EMULSION

| Component | range [% w/w] |
|---|---|
| Aqua | as needed up to 100 |
| Oils (vegetable or synthetic), Waxes, | 5-30 |
| Butters (C10-18 triglycerides, | |
| Butyrospermum parkii butter, Alba | |
| wax, Caprylic/Capric triglyceride) | |
| Silicones (Dimethicone, Dimethicone | 1-10 |
| crosspolymer, dimethiconol) | |
| Wetting agents (Glycerin, propylene | 1-10 |
| glycol, butylene glycol) | |
| Rheology modifiers (Polyacrylate | 1-5 |
| crosspolymer-6, Carbomer, xanthan | |
| gum) | |
| Emulsifiers (C14-22 Alcohols, C12-20 | 0.5-5 |
| Alkyl Glucoside, PEG-100 stearate, | |
| potassium cethyl phosphate) | |
| Preservatives (Potassium sorbate, | 0.1-1.5 |
| sodium benzoate, phenoxyethanol) | |
| Stabilizers (chelators, antioxidants) | 0.1-2 |
| (disodium EDTA, Pentaerythrityl | |
| tetra-di-t-butyl | |
| hydroxyhydrocinnamate, tocopherol) | |
| Perfume | 0.1-1 |
| liquid composition of prot-21 | 0.5-10% |

Formulation 2-SERUM

| Component | range [% w/w] |
|---|---|
| Aqua | as needed up to 100 |
| Oils (vegetable or synthetic), Waxes, | 3-15 |
| Butters (C10-18 triglycerides, | |
| Butyrospermum parkii butter, Alba | |
| wax, Caprylic/Capric triglyceride) | |
| Silicones (Dimethicone, Dimethicone | 1-5 |
| crosspolymer, dimethiconol) | |
| Wetting agents (Glycerin, propylene | 1-10 |
| glycol, butylene glycol) | |
| Rheology modifiers (Polyacrylate | 1-5 |
| crosspolymer-6, Carbomer, xanthan | |
| gum) | |
| Emulsifiers (C14-22 Alcohols, C12-20 | 0.5-5 |
| Alkyl Glucoside, PEG-100 stearate, | |
| potassium cethyl phosphate) | |
| Preservatives (Potassium sorbate, | 0.1-1.5 |
| sodium benzoate, phenoxyethanol) | |
| Stabilizers (chelators, antioxidants) | 0.1-2 |
| (disodium EDTA, Pentaerythrityl | |
| tetra-di-t-butyl | |
| hydroxyhydrocinnamate, tocopherol) | |
| Perfume | 0.1-1 |
| liquid composition as prot-21 | 0.5-10% |

Formulation 3-Detergent

| Component | range [% w/w] |
|---|---|
| Aqua | as needed up to 100 |
| Oils (vegetable or synthetic), Waxes, | 3-15 |
| Butters (C10-18 triglycerides, | |
| Butyrospermum parkii butter, Alba | |
| wax, Caprylic/Capric triglyceride) | |
| Wetting agents (Glycerin, propylene | 1-10 |
| glycol, butylene glycol) | |
| Rheology modifiers (Polyacrylate | 1-5 |
| crosspolymer-6, Carbomer, xanthan | |
| gum) | |
| Surfactants (Lauryl glucoside, | 0.5-5 |
| Laureth-7 citrate, Sodium lauroyl | |
| sarcosinate) | |
| Preservatives (Potassium sorbate, | 0.1-1.5 |
| sodium benzoate, phenoxyethanol) | |
| Stabilizers (chelators, antioxidants) | 0.1-2 |
| (disodium EDTA, Pentaerythrityl | |
| tetra-di-t-butyl | |
| hydroxyhydrocinnamate, tocopherol) | |
| Perfume | 0.1-1 |
| liquid composition as prot-21 | 0.5-10% |

### Formulation 4 O/W Emulsion

Formulation 4 contains the same components and in the same amounts as formulation 1 with the only difference being that the liquid composition of PROT.31 is used in the same amounts as the liquid composition of PROT.21 used in formulation 1.

### Formulation 5 Serum

Formulation 5 contains the same components and in the same amounts as formulation 2 with the only difference being that the liquid composition of PROT.31 is used in the same amounts as the liquid composition 21 of the serum of formulation 2.

### Formulation 6 Detergent

Formulation 6 contains the same components and in the same amounts as formulation 3 with the only difference being that the liquid composition of PROT.31 is used in the same amounts as the liquid composition PROT.21 of the serum of formulation 3.

### Formulation 7 O/W Emulsion

Formulation 7 contains the same components and in the same amounts as formulation 1 with the only difference being that the liquid composition of PROT.40 is used in the same amounts as the liquid composition prot.21 used in formulation 1.

### Formulation 8 Serum

Formulation 8 contains the same components and in the same amounts as formulation 2 with the only difference being that the liquid composition of PROT.40 is used in the same amounts as the liquid composition PROT 21 of the serum of formulation 2.

### Formulation 9 Detergent

Formulation 9 contains the same components and in the same amounts as formulation 3 with the only difference being that the liquid composition of PROT.40 is used in the same amounts as the liquid composition PROT.21 of the serum of formulation 3.

### Formulation 10 O/W Emulsion

Formulation 10 contains the same components and in the same amounts as formulation 1 with the only difference being that the liquid composition of PROT.41 is used in the same amounts as the liquid composition Prot.21 used in formulation 1.

### Formulation 11 Serum

The formulation 11 contains the same components and in the same amounts as the formulation 2 with the only difference being that the liquid composition PROT.41 is used in the same amounts as the liquid composition PROT.21 of the serum of the formulation 2.

### Formulation 12 Detergent

Formulation 12 contains the same components and in the same amounts as formulation 3 with the only difference being that the liquid composition of PROT.41 is used in the same amounts as the liquid composition PROT.21 of the serum of formulation 3.

Comparative Test with Ceramage.

As anticipated in the state of the art of the present disclosure, the commercial product Ceramage produced by the owner of the present patent application contains the same components as the liquid composition as claimed in claim 1, however, at the concentrations used in Ceramage these cannot give rise to liquid compositions.

In fact, in the Ceramage product the concentrations of components a), b) and c) are as follows

Ceramide 0.3%, isopalmide 0.5% and di-(phytosteryl-2-octyldodecyl)-N-lauroyl-L-glutamate 0.5%.

A mixture of 100 g of the above compounds was then prepared which respected the weight ratios of the three compounds in Ceramage.

So the content in this mixture of ceramide was 23 g, that of isopalmide, as well as that of di-(phytosteryl-2-octyldodecyl)-N-lauroyl-L-glutamate was 38.5 g each.

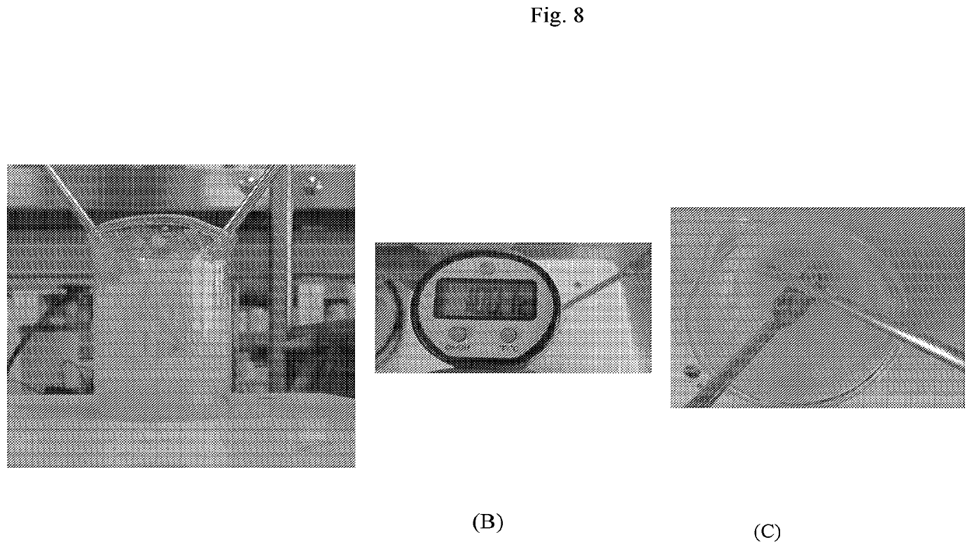

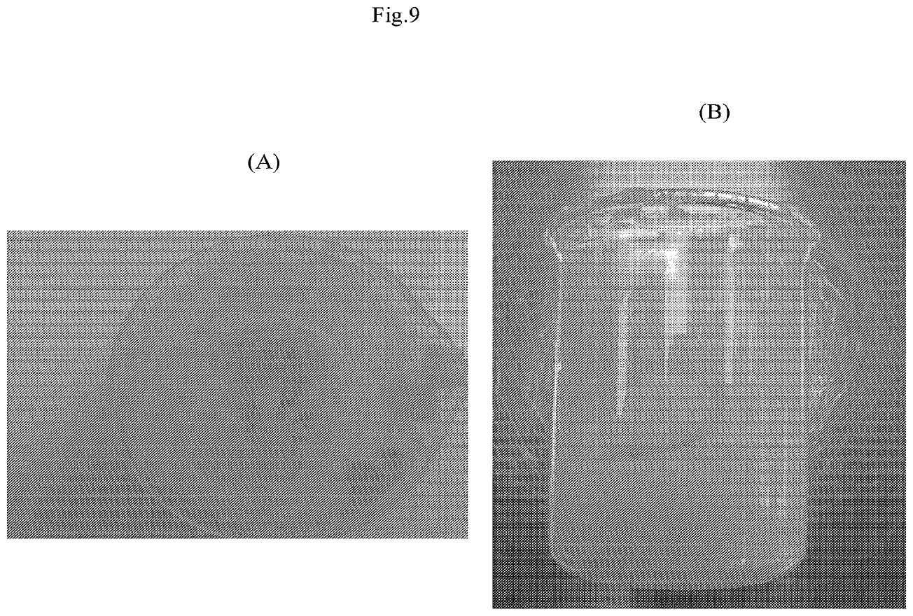

At such high concentrations of isopalmide as well as of ceramide compared to di-(phytosteryl-2-octyldodecyl)-N-lauroyl-L-glutamate, it is not possible to obtain liquid compositions of the aforementioned components, in fact the mixture of these three components, even if kept under magnetic stirring at 100° C., gives rise to a heterogeneous suspension (see FIGS. 8(A) and 8(B)), which once the magnetic stirring is suspended and the temperature is lowered by a few degrees, already tends to solidify (FIG. 8(C)).

After 48 hours at room temperature, the mixture is completely solidified as shown in FIGS. 9(A) and 9(B) in the latter photo air bubbles are visible within the solidified mixture together with crystal formations.

1. Liquid composition consisting of:
a) at least one ceramide
b) at least one palmitic acid amide of formula (I)
CH₃(CH₂)₁₄CONHR¹ (I)
and
c) di-(phytosteryl-2-octyldodecyl)-N-lauroyl-L-glutamate wherein R¹ is a straight or branched alkyl selected from the group consisting of a C1-C6 straight or branched alkyl optionally substituted with a hydroxyl group, a C4-C6 cycloalkyl, and wherein said components a) and b) are dissolved in said component c).

2. Liquid composition according to claim 1, wherein R¹ is selected from: n-propyl, isopropyl, n-butyl, -n-pentyl, iso-pentyl, ethylene-hydroxy, cyclobutyl, cyclopentyl and cyclohexyl.

3. Liquid composition according to claim 1, wherein the palmitic acid amides are selected from N-isopropyl-palmitoyl amide and palmitoylethanolamide or a mixture thereof.

4. Liquid composition according to claim 1, wherein said ceramide or component a) is selected from N-acetylsphyingosine (ceramide 2 or NS) or the amide of phytosphingosine with stearic acid (Ceramide 3 or NP).

5. Liquid composition according to claim 1, wherein each component a) and b) is present in concentrations comprised between 0.05 and 2% by weight on the total weight of said association.

6. Liquid composition according to claim 1, wherein the weight ratio of the components a) and b) is comprised between 1:2 and 2:1.

7. Liquid composition according to claim 1, wherein the weight ratio of the components a) and b) is 1:1.

8. Liquid composition according to claim 1, wherein in component b) both the N-isopropylpalmitoyl amide and the palmitoylethanolamide are present in weight ratios comprised between 1:2 and 2:1.

9. Topical formulation comprising the liquid composition according to claim 1 in amounts comprised between 0.5 and 10% by weight on the total weight of said topical formulation.

10. Topical formulation according to claim 9, wherein said liquid composition is contained in amounts between 1 and 5% by weight, on the total weight of said topical formulation.

11. Topical formulation according to claim 9, in the form of an oil-in-water emulsion and is substantially hydrophilic.

12. Topical formulation according to claim 9, in the form of a water-in-oil emulsion and is substantially lipophilic.

13. Topical formulation according to claim 9, in anhydrous form.

14. Topical formulation for preventing and treating skin inflammatory processes.

15. Topical formulation according to claim 14, wherein said skin inflammatory processes are associated or involved in skin inflammatory diseases or are associated or involved in skin ageing processes.

16. The liquid composition according to claim 4, wherein said ceramide is the amide of phytosphingosine with stearic acid.

17. The liquid composition according to claim 5, wherein each component a) and b) is present in concentrations between 0.1 and 1% by weight.

18. The liquid composition according to claim 8, wherein in component b) the N-isopropylpalmitoyl amide and the palmitoylethanolamide are present in the weight ratio of 1:1.

19. A method for treating and preventing skin inflammatory processes comprising topically administering an effective amount of the topical formulation according to claim 9 to a subject in need thereof, wherein said skin inflammatory processes are associated or involved in skin inflammatory diseases or are associated or involved in skin ageing processes.
